(19) United States
(12) Patent Application Publication
(10) Pub. No.: US 2016/0045373 A1
(43) Pub. Date: Feb. 18, 2016

(54) EAR PLUG WITH CONTAINER

(71) Applicant: Nick Williams, Naples, FL (US)

(72) Inventor: Nick Williams, Naples, FL (US)

(21) Appl. No.: 14/828,105

(22) Filed: Aug. 17, 2015

Related U.S. Application Data
(60) Provisional application No. 62/037,856, filed on Aug. 15, 2014.
(60) Provisional application No. 62/186,934, filed on Jun. 30, 2015.

(51) Int. Cl. A61F 11/10; A61F 11/12

(52) U.S. Cl. CPC A61F 11/10; A61F 11/12

(57) ABSTRACT

An ear protection system may include an ear plug and a storage container. The ear plug may include an elastic component and an inelastic component. The ear plug may be heated, for instance, by filling the storage container with water, at least partially submerging the ear plug in the water filled container, and microwaving the container and the ear plug. The elastic component may then become compressible in order to form to the anatomy of the user's ear. A plurality of indentations may be provided on the elastic component to facilitate the compression. The ear plug may also accommodate electronic features, such as a speaker, as well as a lanyard for holding the ear plugs. The ear plug may be attached to, and used in conjunction with, an electronic device, which may be a communication system such as a wireless headset or speaker.

### CROSS REFERENCE TO RELATED APPLICATIONS

This application claims priority to U.S. Provisional Application No. 62/037,856 filed on Aug. 15, 2014, and U.S. Provisional Application No. 62/186,934 filed on Jun. 30, 2015, the entire contents of which are incorporated herein by reference.

### FIELD OF THE DISCLOSURE

This disclosure relates generally to noise protection devices for human ears often referred to as ear plugs.

### BACKGROUND

Noise protection devices for insertion into the ear canal, more commonly referred to as ear buds or ear plugs, are utilized in a variety of applications. In each application, the ear plug is designed to protect the inner anatomical components of the ear from potentially damaging conditions which the ear may be exposed to. This may be accomplished by inserting a portion of the ear bud into the ear canal thereby blocking a portion of the canal from the potentially damaging environmental condition. Swimmers, for instance, may wear ear buds in order to prevent water from forcefully entering the ear canal thereby causing water-related damage. Similarly, individuals operating in loud environments may wear ear plugs in order to protect the ear from exposure damage due to high-noise levels.

No two ears are alike, however. The size, shape and general dimension varies from individual to individual. An ear bud which cannot securely fit within a portion of the ear canal may fail, either by slipping off or out of the ear, or otherwise not adequately protecting the ear from the potentially damaging environmental condition.

### SUMMARY OF THE DISCLOSURE

The following presents a simplified summary of the disclosure in order to provide a basic understanding of some aspects of the invention. This summary is not an extensive overview of every embodiment disclosed herein. It is intended to neither identify key or critical elements of the various embodiments nor delineate the scope of the disclosure. Its sole purpose is to present some concepts of the disclosure, in accordance with the various embodiments disclosed herein, in a simplified form as a prelude to the more detailed description that is presented later.

In one embodiment of the disclosure, an ear protection system may include an ear plug for insertion into at least a portion of a user's ear, the ear plug including an elastic component which may be heated to a compressible state and an inelastic component which remains in a substantially rigid state as the elastic component is heated to a compressible state, The ear protection system may further include a storage container including a display section having a case dimensioned to hold the ear plug, and a seal with at least a portion of the seal removable so as to expose the ear plug held in the case, and a lid fittable over the seal.

In another embodiment of the disclosure, an ear plug may include an elastic component including a chamber portion having an outer end and an opposed inner end, and a projection portion having a proximate end beginning at or adjacent to the chamber portion and a distal end opposed to the proximate end, the projection portion including a plurality of indentations, and an inelastic component including an internal portion, at least a part of the internal portion is substantially surrounded by the chamber portion of the elastic component, the internal portion and the chamber portion substantially co-axial. The ear plug may be heated to an elevated temperature, the elastic component achieving a compressionable state at the elevated temperature and the inelastic component maintaining a substantially rigid state at the elevated temperature.

In a further embodiment of the disclosure, a method for protecting a user's ear may include providing an ear protection system, which includes an ear plug including an elastic component which may achieve a compressible state when heated to an elevated temperature, and an inelastic component which remains in a substantially rigid state at the elevated temperature, and a storage container including a display section having a case dimensioned to hold the ear plug, filling the case with a fluid while the ear plug is held in the case, thereby at least partially submerging the ear plug within the fluid, heating the fluid to the elevated temperature; compressing the elastic component of the ear plug into the compressed state, inserting the ear plug in its compressed state into the user's ear canal, and allowing the ear plug to cool and conformingly position itself within the user's ear.

The following description and the annexed drawings set forth certain illustrative aspects of the embodiments of the disclosure. These aspects are indicative, however, of but a few of the various ways in which the principles of the disclosure may be employed and the various embodiments are intended to include all such aspects and their equivalents. Other advantages and novel features will become apparent from the following description when considered in conjunction with the drawings.

### BRIEF DESCRIPTION OF THE FIGURES

FIG. 1 illustrates a top view of an embodiment of an ear plug in accordance with the disclosure;

FIG. 2 illustrates a bottom view of the ear plug of FIG. 1;

FIG. 3 illustrates a front view of the ear plug of FIG. 1;

FIG. 4 illustrates a rear view of the ear plug of FIG. 1;

FIG. 5 illustrates an exploded perspective view of an embodiment of an ear plug in accordance with the disclosure;

FIG. 6 illustrates a rear perspective view of the ear plug of FIG. 5 with the breakaway component removed;

FIG. 7 illustrates a rear view of the ear plug of FIG. 5 with the breakaway component removed;

FIG. 8 illustrates a bottom view of the ear plug of FIG. 5 with the breakaway component removed;

FIG. 9 illustrates a bottom cross sectional view of the ear plug of FIG. 5 along cross sectional line 9-9 as shown in FIG. 8;

FIG. 10 illustrates a bottom view of an embodiment of an ear plug in accordance with the disclosure, the elastic component shown semi-transparently;

FIG. 11 illustrates an embodiment of an ear plug shown in an inserted position within the ear canal of a user;

FIG. 12 illustrates an embodiment of an ear protection system including an embodiment of an ear plug held in an embodiment of a storage container, in accordance with the disclosure;

FIG. 13 illustrates the ear plug and storage container of FIG. 12 with the storage container sequentially arranged in an embodiment of a product display;

FIG. 14 illustrates an exploded view of an ear protection system including an embodiment of two ear plugs and an embodiment of a storage container, in accordance with the disclosure;

FIG. 15 illustrates an embodiment of storage container with an insertable heating compartment;

FIG. 16 illustrates an embodiment of a storage container with another embodiment of an insertable heating compartment;

FIG. 17 illustrate an embodiment of a lanyard in accordance with the disclosure;

FIG. 18 illustrates an embodiment of a lanyard connected with an embodiment of an ear plug, in accordance with the disclosure;

FIG. 19 illustrates a front view of an embodiment of an ear plug with an attached embodiment of an electronic device;

FIG. 20 illustrates a side view of the ear plug and attached electronic device of FIG. 19;

FIG. 21 illustrates a side exploded view of an embodiment of an ear plug and an embodiment of an electronic device connectable with the ear plug;

FIG. 22 illustrates a front-perspective view of an embodiment of an ear plug with an attached embodiment of an electronic device;

FIG. 23 illustrates a side cross-section view of the ear plug and attached electronic device of FIG. 22; and

FIG. 24 illustrates an exploded, rear-perspective view of the ear plug and electronic device of FIG. 22.

### DETAILED DESCRIPTION

The following detailed description and the appended drawings describe and illustrate some embodiments of the disclosure for the purpose of enabling one of ordinary skill in the relevant art to make and use these embodiments. As such, the detailed description and illustration of these embodiments are purely illustrative in nature and are in no way intended to limit the scope of the disclosure in any manner. It should also be understood that the drawings are not necessarily to scale and in certain instances details may have been omitted, which are not necessary for an understanding of the embodiments, such as details of fabrication and assembly. In the accompanying drawings, like numerals represent like components.

In one embodiment of the disclosure, an ear protection system may include an ear plug for insertion into at least a portion of a user's ear, the ear plug including an elastic component which may be heated to a compressible state and an inelastic component which remains in a substantially rigid state as the elastic component is heated to a compressible state, The ear protection system may further include a storage container including a display section having a case dimensioned to hold the ear plug, and a seal with at least a portion of the seal removable so as to expose the ear plug held in the case, and a lid fittable over the seal.

In further embodiments, the display section of the storage container may include a fill edge. The seal may be constructed from a substantially metallic material. The seal may a tab operable to puncture the seal and pull away the removable portion of the seal when pulled by a user. The ear plug may include a cap protruding from the inelastic component, and the display section may include a cavity to fittably receive the cap of the ear plug thereby securing the ear plug with a surface of the case. The ear protection system may also include a lanyard, the lanyard including a cord and a securing mechanism, the securing mechanism dimensioned to securely receive the cap of the ear plug. The ear protection system may further include a heating compartment, the heating compartment dimensioned to fit within the case of the display section and having an internal are for filling with a reactant. The reactant may be quicklime. The ear protection system may also include an electronic device having a connector attachable to the inelastic component of the ear plug.

In another embodiment of the disclosure, an ear plug may include an elastic component including a chamber portion having an outer end and an opposed inner end, and a projection portion having a proximate end beginning at or adjacent to the chamber portion and a distal end opposed to the proximate end, the projection portion including a plurality of indentations, and an inelastic component including an internal portion, at least a part of the internal portion is substantially surrounded by the chamber portion of the elastic component, the internal portion and the chamber portion substantially co-axial. The ear plug may be heated to an elevated temperature, the elastic component achieving a compressionable state at the elevated temperature and the inelastic component maintaining a substantially rigid state at the elevated temperature.

In further embodiments, the projection portion of the elastic component may be substantially crescent shaped. At least one indentation of the plurality of indentations may be provided on a first surface of projection portion and at least one matching indentation may be provided adjacent to the first surface on an opposed surface to the first surface. The inelastic component may include an insertion portion, the insertion portion projecting from the internal portion in a direction substantially co-axial with the internal portion and the chamber portion of the elastic component. The elastic component may further include an insertion portion, the insertion portion may project chamber portion in a direction substantially co-axial with the chamber portion and the internal portion of the inelastic component. The inelastic component may further include a cap projecting from the internal portion proximate to the outer end of the elastic component, the cap substantially co-axial with the chamber portion and the internal portion. The inelastic component may be substantially manufactured from Ethylene-vinyl acetate. The elastic component may be substantially manufactured from a thermoplastic elastomer.

In another embodiment of the disclosure, a method for protecting a user's ear may include providing an ear protection system, which includes an ear plug including an elastic component which may achieve a compressible state when heated to an elevated temperature, and an inelastic component which remains in a substantially rigid state at the elevated temperature, and a storage container including a display section having a case dimensioned to hold the ear plug, filling the case with a fluid while the ear plug is held in the case, thereby at least partially submerging the ear plug within the fluid, heating the fluid to the elevated temperature; compressing the elastic component of the ear plug into the compressed state, inserting the ear plug in its compressed state into the user's ear canal, and allowing the ear plug to cool and conformingly position itself within the user's ear.

In further embodiments, the fluid may be heated by placing the case filled with the fluid and holding the ear plug into a microwave and activating the microwave. The fluid may be water and the water may be heated by placing a compartment into the case filled with fluid, the compartment holding quicklime thereby causing a thermal reaction to heat the water.

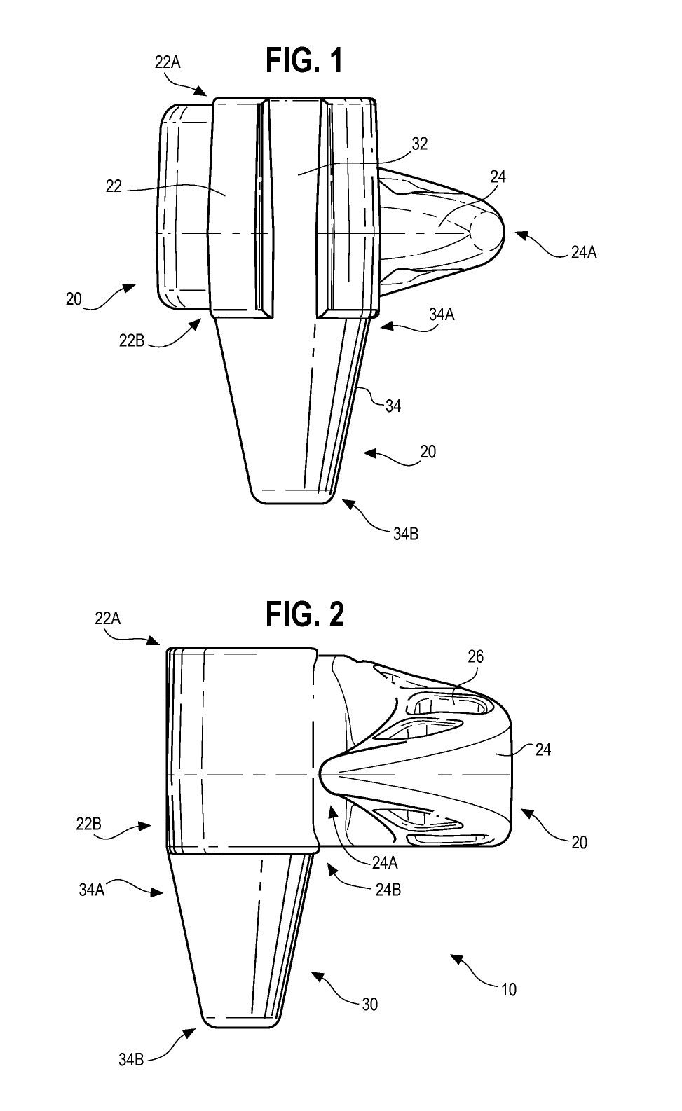

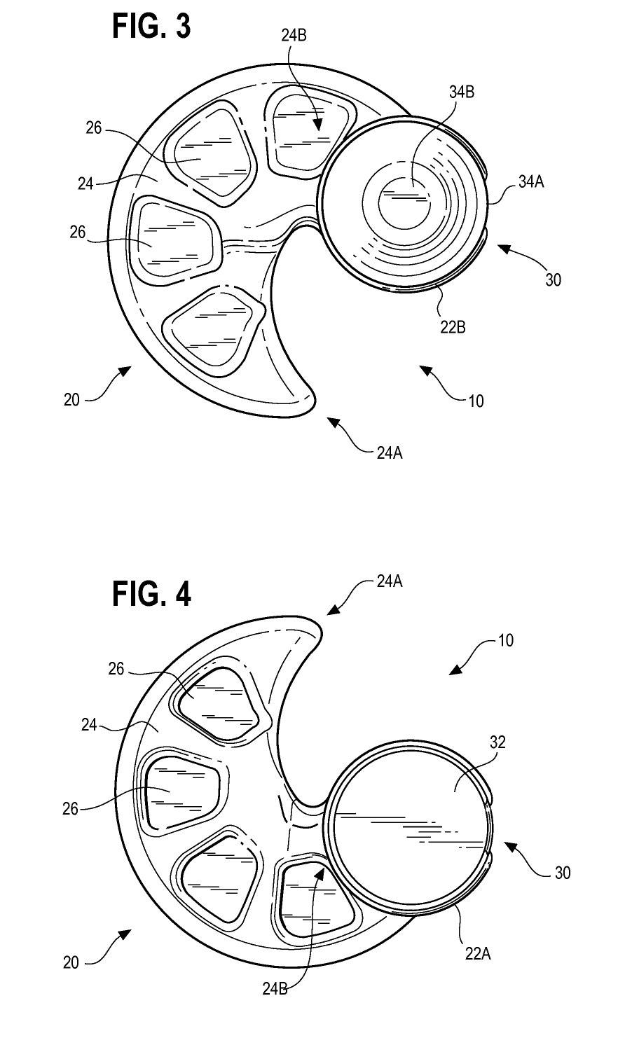

With reference to FIGS. 1-4, an ear plug 10 is provided for protecting the ear canal of a user. Ear plug 10 may be composed of a first, formable component 20 and a second non-formable component 30. Formable component 20 may be elastic and may be compressible for insertion into the cavity of a user's ear, in accordance with embodiments described herein. In contrast, non-formable component 30 may be plastic or inelastic and designed to remain substantially rigid whereas formable component 20 is compressible. Elastic or formable component 20 may include a chamber portion 22 for holding an internal portion 32 of plastic component 30. In some embodiments, all or part of internal portion 32 may be circumferentially surrounded by chamber portion 22, while in other embodiments such as the illustrated embodiment of FIGS. 1-4, only a portion of internal portion 32 may be surrounded.

In one embodiment, elastic chamber 22 may be substantially cylindrical and may include an outer end 22A and an inner end 22B. In addition to chamber portion 22, elastic component 20 may further include a projecting portion 24 which may project away from chamber portion 22. As shown in the illustrated embodiment, projecting portion 24 may be formed into a crescent or semi-circular shape having a first or distal projection end 24A and a second or proximate projection end 24B. Projecting portion 24 may also include a plurality of cavities or indentations 26. Each of the indentations 26 may have a shallow enough depth to permit the existence of indentations on both sides of projecting portion 24, however embodiments where indentations 26 are provided on only the front or rear side are also contemplated within the disclosure. Furthermore, distal end 24A of projecting portion 24 may have a narrower width than the base end 24B of projecting portion 24 proximate to chamber 22, and in one embodiment the distal end 24A of projection portion 24 may come to a point. This change in thickness from proximate end 24B to distal end 24A may constitute a tapering of projecting portion 24.

Ear plug 10 may also include an insertion portion 34 defined between a first or distal insertion end 34B and second or proximate insertion end 34A. The proximate insertion end 34A may be defined adjacent to inner end 22B of chamber portion 22. For the embodiments of FIGS. 1-4, the insertion portion is an extension of internal portion 32 of plastic component 30. Thus, in one embodiment the plastic component 30 may extend from outer end 22A to distal insertion end 34B, with elastic component 20 surrounding at least part of internal portion 32. As shown for instance in some subsequent illustrated embodiments, the insertion portion 34 may alternatively be an extension of elastic component 20.

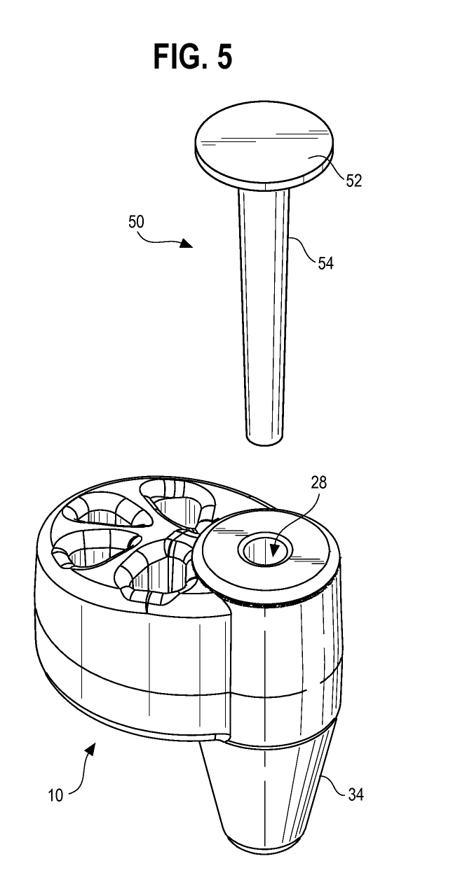

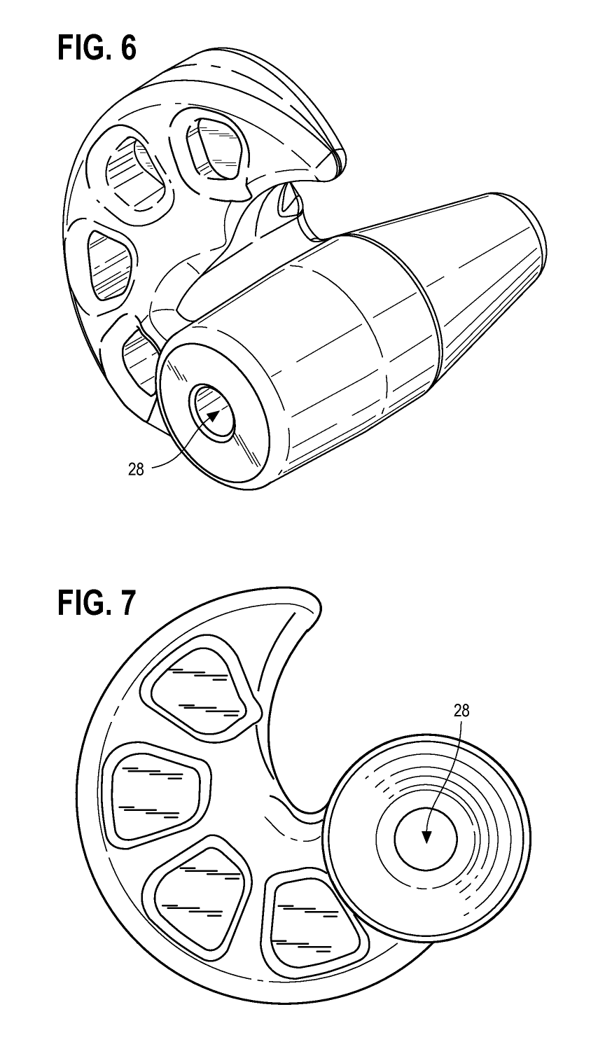

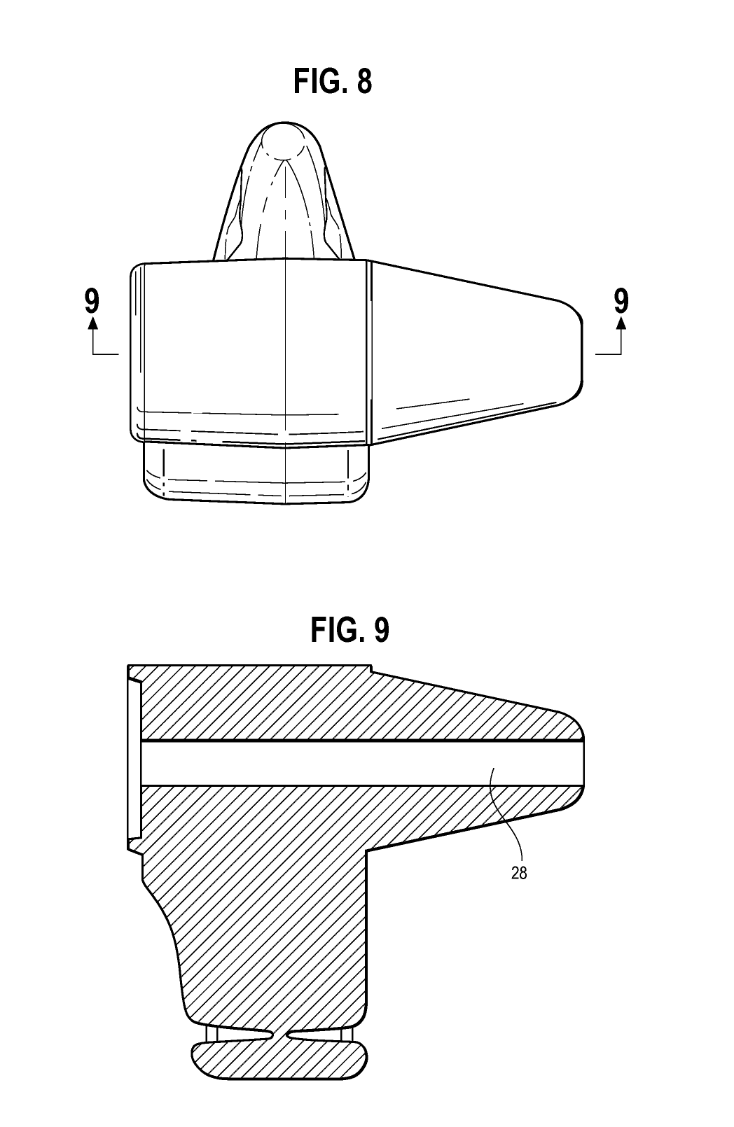

FIGS. 5-9 illustrate another embodiment of ear plug 10 including an insertable component or pin 50 having a head 52 and a stem 54 projecting away from head 52. A distal portion of stem 54 may be insertable into a channel 28 running through at least a portion of chamber 22. In one embodiment, channel 28 radially bisects chamber 22 and extends from outer end 22A to insertion end 34B. Breakaway component 50 may be an overmolded thermoplastic vulcanizate (TPV) material. A user of ear plug 10 may pull on breakaway component 50 thereby removing breakaway component 50 from chamber 22 in order to reveal channel 28. With channel 28 revealed, various items or materials may be introduced into channel 28, for instance audio amplifying devices such as electronic ear phones or filters of various noise reduction. By extending channel 28 to insertion end 34B, the unwanted sound may be blocked from the ear canal while still permitting selective sound to be transmitted into the ear canal through ear phones provided in or near channel 28. Other items may be stored within, or inserted into, chamber 28. In some embodiments, chamber 28 is defined through plastic component 30, while in other embodiments plastic component 30 may be limited to projecting portion 34 while elastic component 20 defines chamber 32, and channel 28 is defined through elastic component 20.

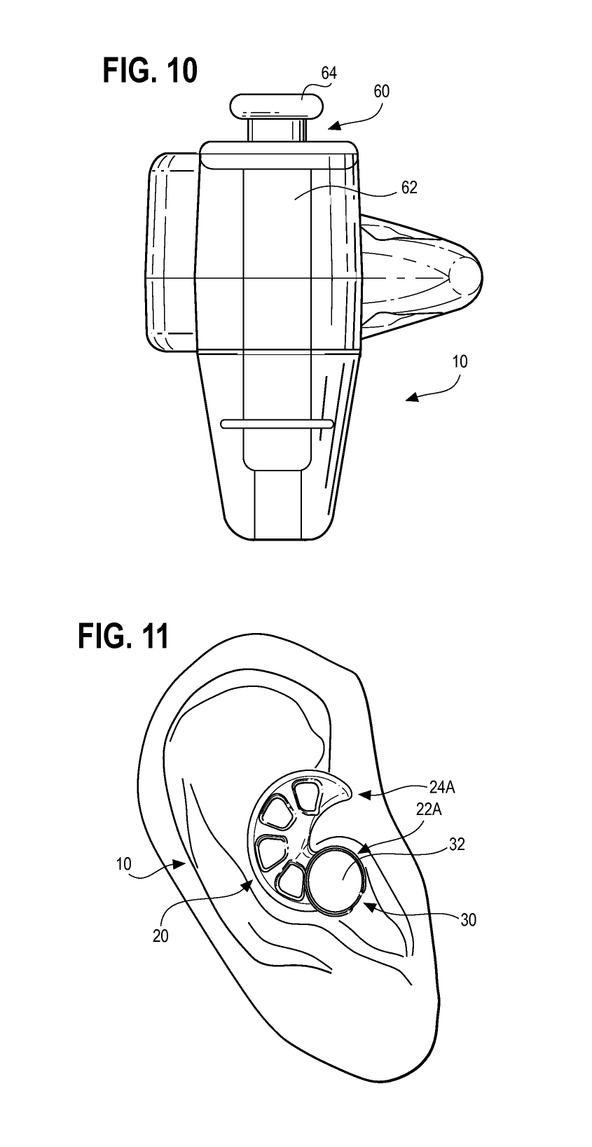

FIG. 10 illustrates an additional embodiment of ear plug 10 where at least the outer surface of insertion portion 34 is constructed from formable material 20, and the informable portion 30 includes an insertion member 60. As illustrated, insertion member 60 may include an internal portion 62 substantially surrounded by formable elastic material 20, and a cap 64 provided substantially external from the formable material 20. This embodiment of ear plug 10 may allow for communication systems or devices to be used with the ear plug 10 including but not limited to BLUETOOTH, headphones, earbuds, or headsets. Additional embodiments of ear plug 10 as may utilized with electronic devices or communication systems are discussed in further detail below. Because of the difference between the thermal properties of formable portion 20 and informable portion 30, while formable portion may be heated 20 to be made compressible, the cap 64 of the informal portion 30 may not necessarily be similarly heated and, thus, a user may grab 64 to avoid burning from touching formable portion 20, especially if the formable portion 20 was overheated.

In the various embodiments of ear plug 10, plastic component 30 may be manufactured from EVA (Ethylene-vinyl acetate), polyester material, such as polycaprolactone resin, compounded under heat. Such a compounding made in a double shear compounder or a similar mechanism, and the polyester resin may be combined with calcium carbonate and or glass bubbles as well as coloring agents or pigments. This resulting polyester based compound may be next formed into pellets for molding as part of the manufacturing process. The resulting compound may be mixed with additional polyester material as a master batch or can alternatively be used directly in molding depending on the desired color or cooling concentrations. The mixed compound may then be fed into an injection molding machine with variable parameters. Example parameters associated with the injection molding machine may include a barrel temperature of 120-220° F., a pressure of approximately 1500 psi at 50% speed, and a charging pressure of approximately 1000 psi. The material may then be injected into a material mold having superior heat conducting properties as the injected material. The plastic component may be thereby formed into a chamber held portion 32 and a chamber projection portion 34, or formed into an insertion member 60 with an internal portion 62 and cap 64. As one example of manufacturing, embodiments featuring a projecting portion 34 may begin with initially shaping projection portion 34 into a cone having a wider base end 34A than an insertion end 34B, so as to accommodate insertion in the user's ear canal. Once cooled, the formed plastic component 30 may then be prepared for its combination with elastic component 20, as further described herein.

Elastic component 20 may be formed from an elastic material including a thermoplastic elastomer, such pololefin blend, elastomeric alloys, syternic block copolymers, or other known or to be developed thermoplastic elastomers, and potentially as well as silicone, rubber or other elastic material. This elastic material may be injected over or around the formed plastic component 30, namely around chamber portion 32 or internal portion 62, depending on the embodiment.

FIG. 11 illustrates a method of using ear plug 10, where insertion end 34B is inserted into the ear canal of a user. Prior to insertion of insertion end 34B into a user's ear, elastic component 20 may be heated in of the variety of ways described herein. In one embodiment, a user may dip ear plug 10, and more particularly elastic component 20, into a hot fluid such as hot or boiling water. In one embodiment, ear plug 10 may be submerged in a container of water, and as the water is heated, by a microwave or stove top for instance, the elastic component 20 enters into a conformable or compressible state. This elevation in temperature may then permit reformation of elastic component 20 to compress and form to the user's ear canal upon insertion of insertion portion 34. Such a reformation may permit elastic portion 20 to compress as insertion end 34B is inserted into the ear canal. Elastic component 20 may then reform from its original crescent shape into a conforming shape with the user's outer ear, which may include conforming to the shape defined, at least in part, by the concha and antihelix of the ear. Cavities 26 may assist in the compression of formable component 20 by creating and providing pockets of missing material from which the remaining elastic material may compress into. While projecting portion may operate to block sound, fluid, or any other undesirable material or objects during operation of ear plug 10, the conformed placement of formable component 20 may further assist in blocking sound, fluid, or any other undesirable material or objects from entering ear canal 10. For instance, formable component may operate to absorb sound thereby increasing the effectiveness of ear plug 10 operating as a sound protection device for the user's ear. While inserted, the chamber portion 32 of plastic component 30 may be substantially planar at or near outer end 22A in order to accommodate an advertisement, logo, symbol, or other message which may be seen by a third party viewing the ear plug 10 as worn by a user. A handle or extrusion, such as cap 64, may also be provided at or proximate to outer end 22A.

Additional embodiments of ear plug 10 may include electronic noise canceling transistors molded into plug 10. Additives may also be included in the manufacture of either formable component 20 or plastic component 30 in order to adjust material characteristics of the components. For instance, micro glass and hollow glass spheres, also referred to as glass bubbles, may be added to increase the noise reduction rating of plug 10. In one embodiment, the glass bubbles may be in the approximately 17 micron range with a crush strength at or near 28,000 psi. Such an additive may assist in the molding process, the noise reduction range, or both. Furthermore, while elastic component 20 has an elevated temperature, as may be performed by a user as described herein, the user may cut or otherwise remove excess material inhibiting a tight or comfortable formation to the user's outer ear.

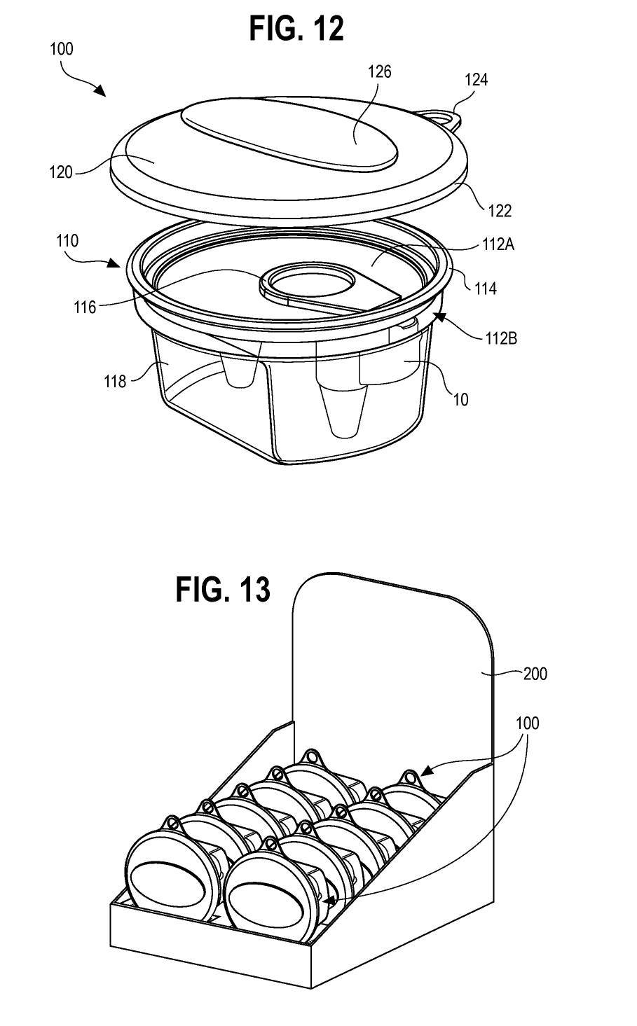

FIGS. 12-14 illustrate embodiments of a storage container 100 for holding embodiments of ear plug 10. Storage container 100 may be composed of a display section 110 joinable with a lid section 120. Display section 110 may include a seal 112 with lid side surface 112B and a substantially opposed display side surface 112B, with an outer perimeter 114 defined there between and at the edge of the seal 112. In one embodiment, seal 112 is substantially circular and formed from a metallic or substantially metallic material. By constructing seal 112 with round and smooth surfaces, such as a conforming perimeter 114 into a circular or oval dimension, then container 100 may be safely used in a microwave, which is one method to heat a fluid as described herein, even though seal 112 includes metallic or substantially metallic material.

Lid 120, which may have an outer perimeter 122 substantially corresponding with the display outer perimeter 114. Lid outer perimeter 122 may then snap fit over display outer perimeter 114, or in another embodiment display outer perimeter 114 may snap fit over lid outer perimeter 122. Lid 120 may be removed, for instance, by grasping a handle 124 protruding from a portion of lid 120. In the illustrated embodiment, handle 124 protrudes from an area proximate to outer perimeter 122. A design surface 126 may also be provided so as to display a logo, symbol, text, or other design on a top surface of lid 120.

A display case 118 may be secured to the display side surface 112B. Case 118 may be sized and dimensioned to hold an embodiment of ear plug 10 between display case 118 and display side surface 112B. Case 118 may also be constructed of a substantially transparent material so that a stored ear plug 10 may be viewable from outside container 100. In order to access a stored ear plug 10, a tab 116 may be provided on lid side surface 112A. Tab 116 may be lifted in order to puncture a seal between outer perimeter 114 and surfaces 112A/112B thereby permitting a user to pull away a portion of the material and access ear plug 10 stored within display case 118.

With particular reference to the embodiment illustrated in FIG. 14, one embodiment of display case 118 may include one or more hollowed-out spaces or cavities 128 at the bottom edge of the display case 118. The hollowed-out spaces may allow an embodiment of ear plug 10 to be secured to display case, for instance by insertion of tip head 55 or cap 64 into hollowed-out space 128. Where two ear plugs 10 are to be stored in case 118, then two cavities 128 may be provided to accommodate secure storage of both plugs 10. Additionally, a fill edge or shelf 130 may be provided on an interior side of case 118. Fill edge 130 may laterally or radially project away in order to define the fill edge or shelf 130 Water or another heatable fluid may be filled up fill edge 130, which would be placed at a dimension substantially corresponding to a length of held plug(s) 10. Thus, filling the case 118 with fluid up to fill edge 130 would correspond with a fill line sufficiently submerging held plug(s) in the fluid.

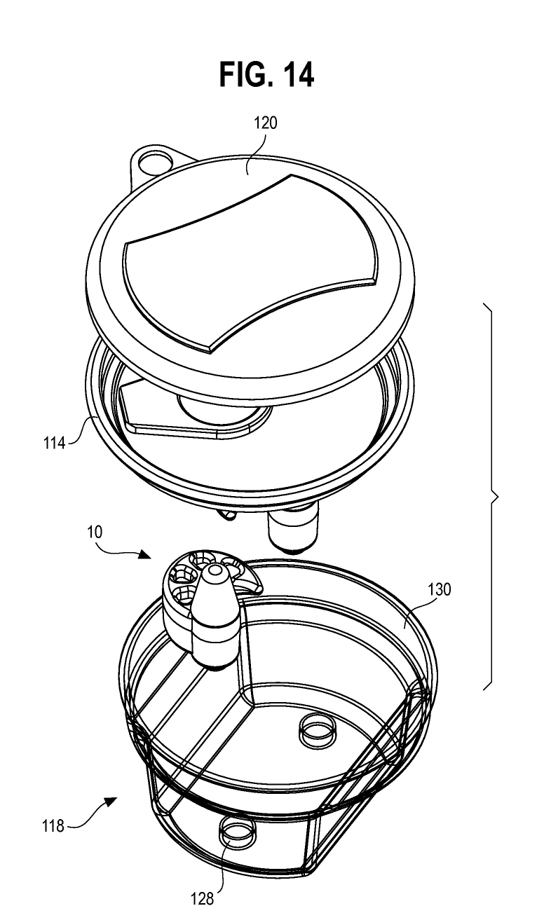

A display stand 200, one embodiment of which is shown in FIG. 13, may hold a plurality of display cases 100. In one embodiment, at least ten (10) display cases 100 are held in a linear arrangement for selection by a user, with design surface 126 presented outwardly as most visible by a potential user.

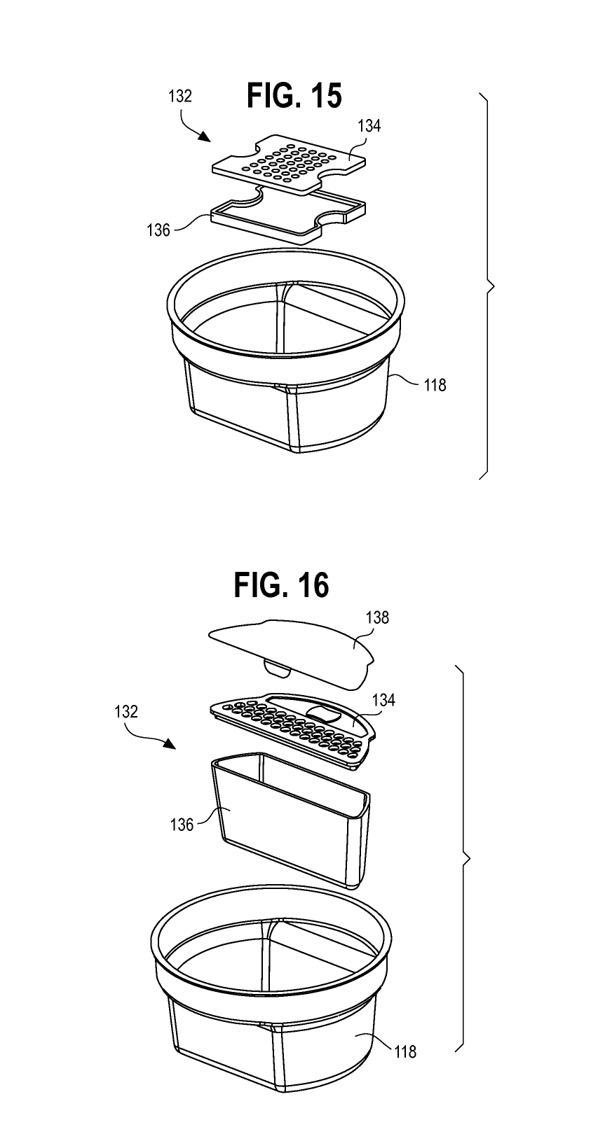

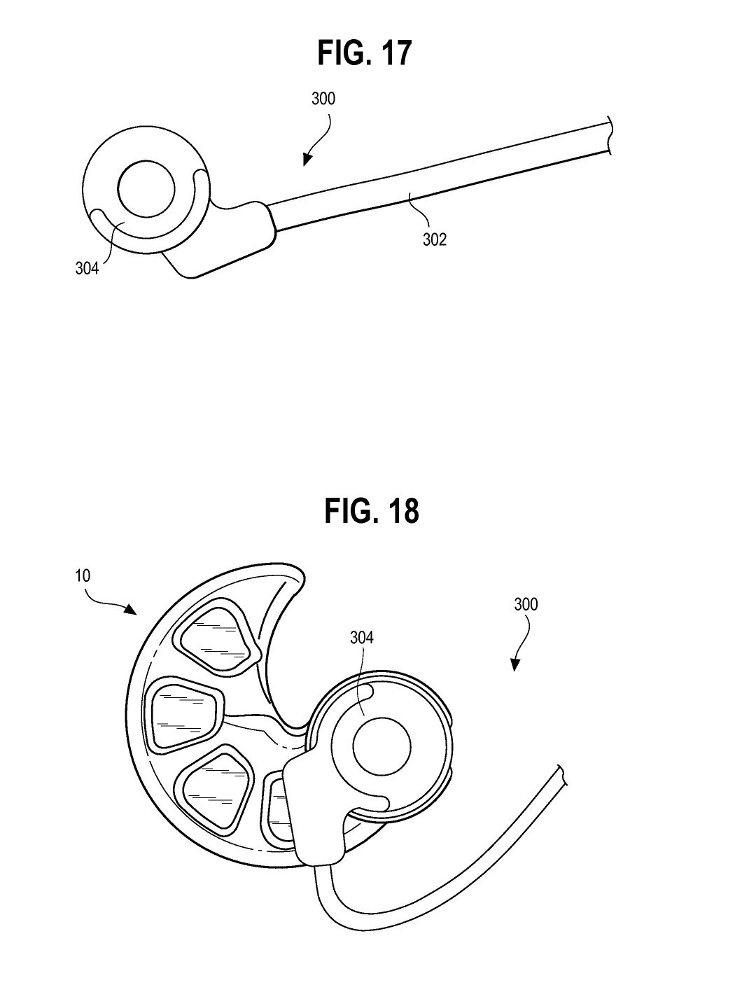

As shown in the illustrated embodiments of FIGS. 15 and 16, storage container 100 may further include a heating compartment 132 insertable within display case 118. The insertable heating compartment 132 may be infused with thermal inducing material. In one embodiment, compartment 132 may be infused with calcium oxide, commonly referred to as quicklime or burnt lime. Adding water to compartment 132 with infused material, such as quicklime, may then create an exothermal reaction for heating ear plug 10. The insertable compartment 132 may include a bottom portion 134 joinable with an upper portion 136, with a plurality of holes through the bottom portion, upper portion, or both. In one embodiment, shown for instance in FIG. 15, compartment 132 may be formed into a relatively thin pad. In another embodiment, shown for instance in FIG. 16, bottom portion 136 may conformingly dimensioned to forcibly fit within an internal portion of case 118. Further, a removable sticker or sealing surface 138 may be provided over upper portion 134, bottom portion 136, or both. Sticker 138 may therefore keep quicklime or other material stored in bottom portion 134 from spilling during transportation of storage container 100. Removal of sticker 138 may thereby expose the internal area of bottom portion 136, allowing water to be added thereby initiating the exothermal reaction.

FIGS. 17 and 18 illustrate an embodiment of a lanyard 300 for securing embodiments of ear plugs 10. The lanyard 300 may be comprised of a cord 302 and a securing mechanism 304 attached at both ends of the cord 302. The cord 302 may also be a rope, string or wire. The cord 302 may be manufactured from polyester, nylon, satin, silk, polyethylene terephthalate, or leather. The securing mechanisms 304 at both ends of the cord 302 may include a ring, hoop, or hook that attaches to the tip 53 or the cap 64 of ear plug 10. Securing the lanyard 300 to each of the ear plugs 10 prevents the ear plugs 10 becoming separated when not secured inside the storage container 100. Furthermore, securing the lanyard 300 to each of the ear plugs 10 allows a user to position the lanyard 300 with attached ear plugs 10 around a neck or shoulder when not inside ears. A secured lanyard 300 may facilitate the user to manipulate the position of ear plugs 10, particularly after the ear plug has been heated to an elevated temperature and has been compressed. Accordingly, lanyard 300 may allow for rotation and repositioning of a formed ear plug 10.

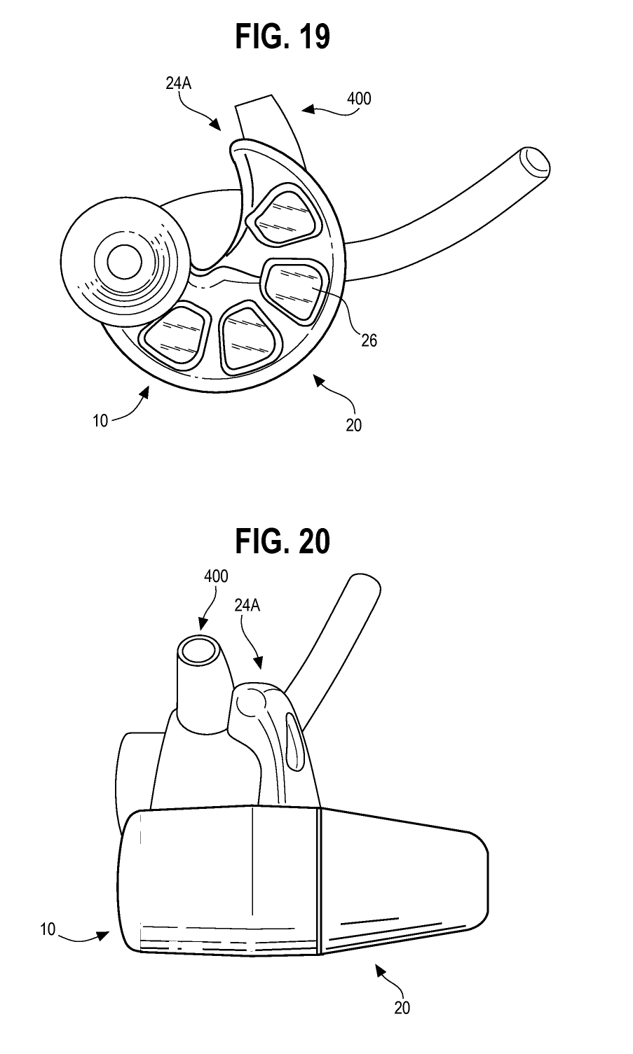

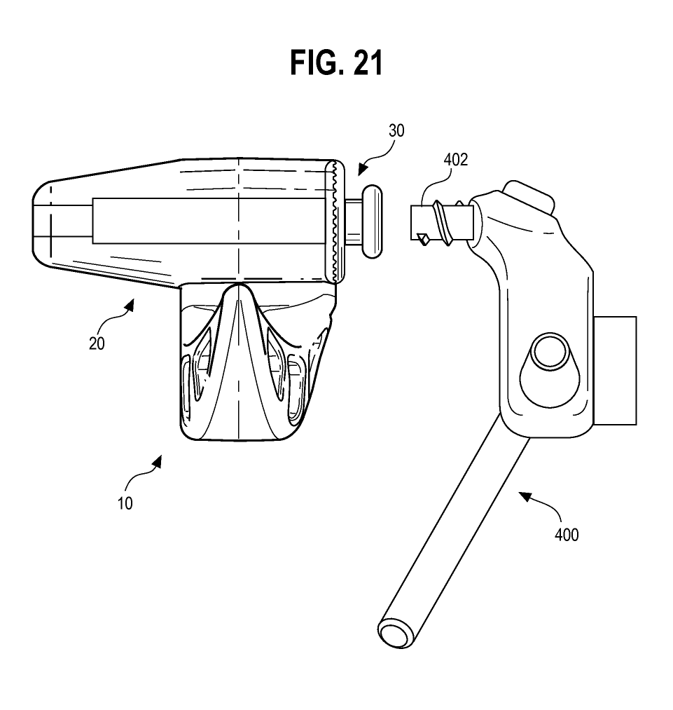

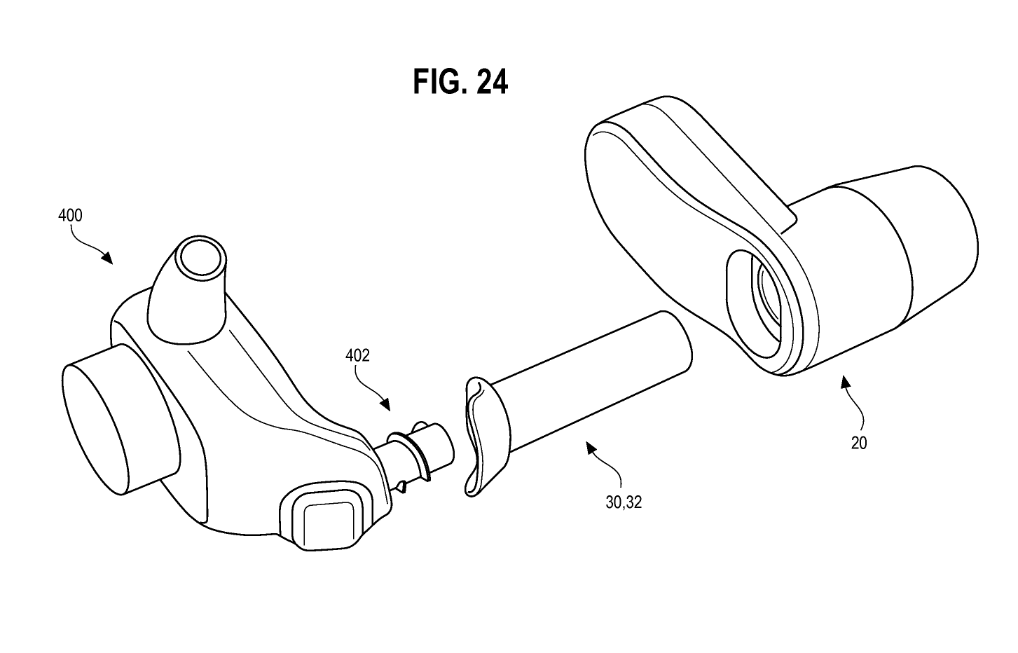

Turning now to FIGS. 19-24, various embodiments of ear plugs 10 may be attached to, and used in conjunction with, electronic devices 400. FIGS. 19-20 and FIG. 21 illustrate embodiments of ear plug 10 having similar features to those previously disclosed herein. An electronic device 400 is shown as being connectable with ear plug 10. Electronic device 400 may have an electronic device connector 402, which in one embodiment may be a threaded protrusion dimensioned and shaped to attach to plastic component 30. In particular, the connector 402 may be at least partially insertable into chamber portion 22. Internal threading may be provided in chamber portion 22 in order to engage matable threading with connector 402. Alternatively, connector 402 may be secured in a press-fitting and held by friction. Other known or to be discovered attachments or connectors are contemplated within the disclosure for holding electronic device 400 with ear plug 10.

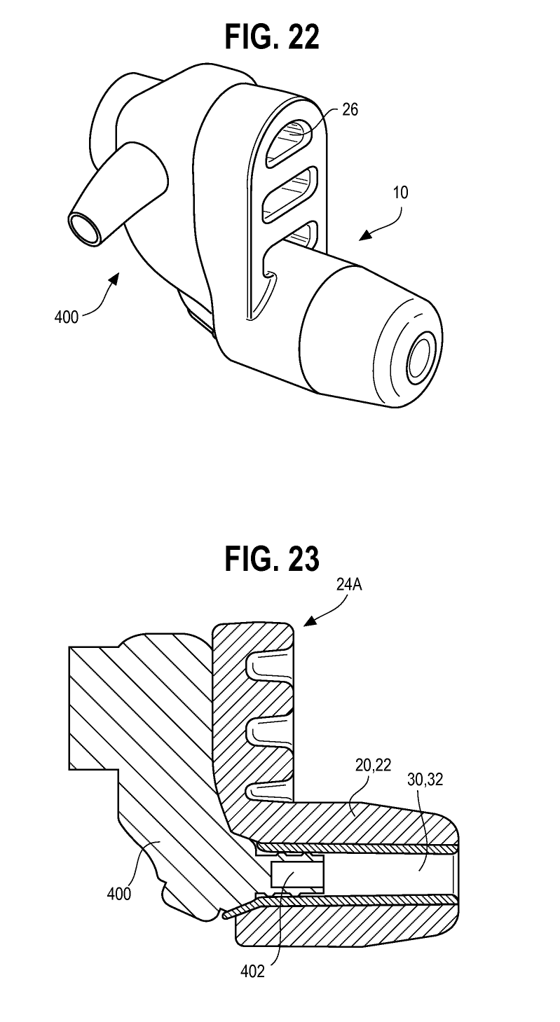

FIGS. 22-24 illustrate an additional embodiment of ear plug 10 attachable with an embodiment of an electronic device 400. Elastic component 20 may include a tunnel portion 22 and a protecting portion having one or more indentations 26. As shown, plastic component 30 may be removably insertable within elastic component 20, including for instance the insertion of internal portion 32 into tunnel portion 22. A connector 402 of device 400 may be insertable into internal portion 32, and held within internal portion 32 for instance by friction push-fit, by matable threads, or any other known or to be developed connector or attachment. The internal portion 32 may be substantially hollow throughout to permit sound passage from device 400 to pass to the user's ear. In this regard, a speaker may be incorporated into, or provided adjacent to, connector 402 so that sound emitted from the speaker may be directed into internal portion 32.

Electronic device 400 may be an communication system or devices to be used with the ear plug 10 including but not limited to BLUETOOTH, headphones, earbuds, or headsets. Accordingly, transmitters, antennas, or other wireless communication technology may be incorporated into device 400. Speakers may also be provided so that a user wearing ear plug 10 may be able to hear sounds from device 400 as may be transmitted from an external source. In this regard, the user may be able to hear only select sounds emitted from device 400 while the user's ear canal is protected by the ear plug 10 from noise pollution. For instance, in the application of environments saturated with damaging noise, such as a shooting range or an airport tarmac, a user may utilize ear plug 10 to protect the user's ear while still being able to hear select noise emitted from device 400, such as music or a telephone call transmitted from the user's smartphone. Electronic device 400 may be constructed from any known or to be developed material suitable for constructing a device 400 as described and utilized herein.

The descriptions set forth above are meant to be illustrative and not limiting. Various modifications of the embodiments, in addition to those described herein, will be apparent to those skilled in the art from the foregoing description. Such modifications are also intended to fall within the scope of the concepts described herein. Each patent, patent application and publication cited or described in this document are hereby incorporated herein by reference, in their entireties.

The foregoing description of possible implementations consistent with the present disclosure does not represent a comprehensive list of all such implementations or all variations of the implementations described. The description of some implementation should not be construed as an intent to exclude other implementations. For example, artisans will understand how to implement the embodiments in many other ways, using equivalents and alternatives that do not depart from the scope of the disclosure. Moreover, unless indicated to the contrary in the preceding description, none of the components described in the implementations are essential to the embodiments disclosed. It is thus intended that the embodiments be considered as illustrative, with a true scope and spirit of the disclosure being indicated by the following claims.

What is claimed is:

1. An ear protection system comprising:
an ear plug for insertion into at least a portion of a user's ear, the ear plug including
an elastic component which may be heated to a compressible state, and
an inelastic component, which remains in a substantially rigid state as the elastic component is heated to a compressible state; and
a storage container including
a display section having a case dimensioned to hold the ear plug, and a seal with at least a portion of the seal removable so as to expose the ear plug held in the case, and
a lid fittable over the seal.

2. The ear protection system of claim 1, the display section of the storage container further including a fill edge.

3. The ear protection system of claim 1, wherein the seal is constructed from a substantially metallic material.

4. The ear protection system of claim 1, wherein the seal has a tab operable to puncture the seal and pull away the removable portion of the seal when pulled by a user.

5. The ear protection system of claim 1, the ear plug further including a cap protruding from the inelastic component, and the display section further including a cavity to fittably receive the cap of the ear plug thereby securing the ear plug with a surface of the case.

6. The ear protection system of claim 5 further comprising a lanyard, the lanyard including a cord and a securing mechanism, the securing mechanism dimensioned to securely receive the cap of the ear plug.

7. The ear protection system of claim 1 further comprising a heating compartment, the heating compartment dimensioned to fit within the case of the display section and having an internal are for filling with a reactant.

8. The ear protection system of claim 1, wherein the reactant is quicklime.

9. The ear protection system of claim 1, further comprising an electronic device having a connector attachable to the inelastic component of the ear plug.

10. An ear plug comprising:
an elastic component including
a chamber portion having an outer end and an opposed inner end, and
a projection portion having a proximate end beginning at or adjacent to the chamber portion and a distal end opposed to the proximate end, the projection portion including a plurality of indentations, and
an inelastic component including an internal portion, at least a part of the internal portion is substantially surrounded by the chamber portion of the elastic component, the internal portion and the chamber portion substantially co-axial,
wherein the ear plug may be heated to an elevated temperature, the elastic component achieving a compressionable state at the elevated temperature and the inelastic component maintaining a substantially rigid state at the elevated temperature.

11. The ear plug of claim 10, wherein the projection portion of the elastic component is substantially crescent shaped.

12. The ear plug of claim 10, wherein at least one indentation of the plurality of indentations is provided on a first surface of projection portion and at least one matching indentation is provided adjacent to the first surface on an opposed surface to the first surface.

13. The ear plug of claim 10, the inelastic component further including an insertion portion, the insertion portion projecting from the internal portion in a direction substantially co-axial with the internal portion and the chamber portion of the elastic component.

14. The ear plug of claim 10, the elastic component further including an insertion portion, the insertion portion projecting chamber portion in a direction substantially co-axial with the chamber portion and the internal portion of the inelastic component.

15. The ear plug of claim 14, the inelastic component further including a cap projecting from the internal portion proximate to the outer end of the elastic component, the cap substantially co-axial with the chamber portion and the internal portion.

16. The ear plug of claim 10 wherein the inelastic component is substantially manufactured from Ethylene-vinyl acetate.

17. The ear plug of claim 10, wherein the elastic component is substantially manufactured from a thermoplastic elastomer.

18. A method for protecting a user's ear, the method comprising:
providing an ear protection system, which includes
an ear plug including an elastic component which may achieve a compressible state when heated to an elevated temperature, and an inelastic component which remains in a substantially rigid state at the elevated temperature, and
a storage container including a display section having a case dimensioned to hold the ear plug;
filling the case with a fluid while the ear plug is held in the case, thereby at least partially submerging the ear plug within the fluid;
heating the fluid to the elevated temperature;
compressing the elastic component of the ear plug into the compressed state;
inserting the ear plug in its compressed state into the user's ear canal; and
allowing the ear plug to cool and conformingly position itself within the user's ear.

19. The method of claim 18, wherein the fluid is heated by placing the case filled with the fluid and holding the ear plug into a microwave and activating the microwave.

20. The method of claim 18, wherein the fluid is water and the water is heated by placing a compartment into the case filled with fluid, the compartment holding quicklime thereby causing a thermal reaction to heat the water.
